(19) United States
(12) Patent Application Publication
(10) Pub. No.: US 2024/0125798 A1
(43) Pub. Date: Apr. 18, 2024

(54) METHOD FOR SCREENING FUNCTIONAL MATERIAL PALLIATIVE OF ALLERGY, ATOPY, AND ITCHINESS

(71) Applicant: SHEBAH BIOTECH INC., Chuncheon-si (KR)

(72) Inventors: Yong-wan CHO, Chuncheon-si (KR); Hye-won LIM, Chuncheon-si (KR); Han-wool JE, Chuncheon-si (KR); Young-bin KIM, Chuncheon-si (KR); Gyu-ri KYEONG, Chuncheon-si (KR); Young-sook KIM, Daejeon (KR)

(21) Appl. No.: 18/278,121

(22) PCT Filed: Nov. 17, 2021
(86) PCT No.: PCT/KR2021/016860

§ 371 Date: Aug. 21, 2023

(30) Foreign Application Priority Data
Feb. 24, 2021 (KR) 10-2021-0024914
Nov. 17, 2021 (KR) 10-2021-0158188

(51) Int. Cl. G01N 33/68; G01N 33/50; G01N 33/58

(52) U.S. Cl. CPC G01N 33/6854; G01N 33/5008; G01N 33/582

(57) ABSTRACT

A method for screening a functional material according to the present invention comprises steps of: treating a linker material-coated substrate with a capture protein; treating the capture protein-treated substrate with a functional material candidate; treating the functional material candidate-treated substrate with biotin-conjugated IgE; and treating the biotin-bound IgE-treated substrate with fluorescently labeled streptavidin.

### TECHNICAL FIELD

The present invention relates to a method for screening a functional material having an activity of alleviating allergy, atopy and itching.

### BACKGROUND ART

Recently, people with sensitive skin who complain of discomfort due to frequent skin troubles caused by fine dust, yellow dust, and rapid changes in seasons and lifestyles have increased rapidly. About 30 to 40% of the total population feels uncomfortable, and it has been reported that 55% of South Korean women and 38.9% of South Korean men feel uncomfortable.

Sensitive skin feels irritable, which results from differences in the distribution, number or sensitivity of skin nerves, or from deterioration in barrier function. Nerve fibers such as A5 and C fibers are associated with skin irritation and involved in mild discomfort in the skin, such as stinging and burning sensation. In addition, deterioration in barrier function is one of the characteristics frequently observed in sensitive skin, and causes irritation as well as redness, allergy, etc.

In addition, South Korea has the highest ratio of the plastic surgery population to the population (1 out of 77 people) in the world, and there is a need to develop care products for a skin that has become sensitive after plastic surgery and cosmetic procedures.

Meanwhile, for the treatment of atopic dermatitis, which is a representative disease of sensitive dermatitis, steroidal agents, immunosuppressants, antibiotics, etc., are used, but they have a disadvantage in that they may be used only for a short period of time because they cause side effects such as abnormal immune function when used for a long period of time.

Therefore, there is a need to develop an effective and safe material capable of alleviating allergy, atopy, and skin itching with less toxicity and side effects.

### PRIOR ART DOCUMENTS

### Patent Documents

Korean Patent No. 10-1600333

### DISCLOSURE

### Technical Problem

An object of the present invention is to provide a method for screening a material capable of alleviating allergy, atopy and skin itching.

### Technical Solution

A method of screening a functional material according to one embodiment of the present invention may comprise steps of: treating a linker material-coated substrate with a capture protein; treating the capture protein-treated substrate with a functional material candidate; treating the functional material candidate-treated substrate with biotin-conjugated IgE; and treating the biotin-conjugated IgE-treated substrate with fluorescently labeled streptavidin.

In one embodiment of the present invention, the method may further comprise, after the step of treating the substrate with the capture protein, a step of treating the substrate with BSA to block non-specific binding.

In one embodiment of the present invention, the method may further comprise, before the step of treating the substrate with the fluorescently labeled streptavidin, a step of washing and drying the biotin-conjugated IgE-treated substrate.

In one embodiment of the present invention, the method may further comprise, before the step of treating the substrate with the functional material candidate, a step of washing and drying the capture protein-treated substrate.

In one embodiment of the present invention, the method may further comprise, after the step of treating the substrate with the fluorescently labeled streptavidin, a step of analyzing the degree of binding between the IgE and the capture protein.

In one embodiment of the present invention, the capture protein may be FcεR1.

### Advantageous Effects

The screening method according to the embodiments of the present invention has the advantage of being able to easily confirm whether the target protein IgE actually binds to its receptor, by directly analyzing the interaction therebetween, and has the effect of showing high efficiency at a relatively low cost compared to other methods such as ELISA plate-based assay.

### BRIEF DESCRIPTION OF DRAWINGS

FIG. 1 schematically shows protein binding that is used in the screening method of the present invention.

FIG. 2 depicts a table and graph showing the results according to Experimental Example 1 of the present invention.

FIG. 3 depicts a table and graph showing the results according to Experimental Example 2 of the present invention.

FIG. 4 is a graph showing the results according to Experimental Example 3 of the present invention.

### BEST MODE

Unless otherwise defined, all terms used herein, including technical or scientific terms, have the same meanings as commonly understood by those skilled in the technical field to which the present invention pertains. Terms such as those defined in generally used dictionaries should be interpreted as having meanings identical to those specified in the context of related technology. Unless definitely defined in the present application, the terms should not be interpreted as having ideal or excessively formative meanings

The present invention provides a method of screening a functional material, the method comprising steps of: treating a linker material-coated substrate with a capture protein; treating the capture protein-treated substrate with a functional material candidate; treating the functional material candidate-treated substrate with biotin-conjugated IgE; and treating the biotin-conjugated IgE-treated substrate with fluorescently labeled streptavidin.

When IgE, the main immune antibody that causes an allergic reaction in the human body, binds to IgE receptor (FcεR1) present on the surface of mast cells (dendritic cells), histamine is released through an intracellular signaling process, resulting in several stimuli. That is, when a material capable of interfering with the interaction between IgE and FcεR1 is discovered, it may be used as an active ingredient for preventing, alleviating or treating atopic dermatitis.

In particular, even if a specific allergen enters and binds to IgE, if IgE can be blocked from binding to FcεR1, allergic reactions as well as atopic dermatitis will not appear.

The inflammation inhibition analysis method, which is a previously developed method for evaluating the efficacy of alleviating skin sensitivity, is performed using a technique of measuring the inhibition of nitric oxide (NO) production and secreted inflammation-related factors (INF-γ, IL-1β, etc.) by an ELISA method. This ELISA method can analyze up to 20 samples at a cost of more than 600,000 to 900,000 won using a kit, but this method has a problem in that it is expensive to analyze a plurality of samples at once.

The method of screening a functional material according to the present invention is characterized in that it can quickly and conveniently analyze whether the target protein IgE actually binds to its receptor, by directly analyzing the interaction therebetween. In addition, the screening method of the present invention has an advantage in that a whole human protein that can actually act in vivo is used instead of a peptide, so that the in vitro activity of the protein can be actually equally applied to the in vivo activity.

In addition, the screening method of the present invention has an advantage in that it may be miniaturized and highly integrated, and thus can show high efficiency at a relatively low cost compared to other methods such as ELISA plate-based method.

That is, when the method of screening a functional material according to the present invention is used, there are advantages in that it is possible to analyze nearly 400 samples at once, and accurate analysis results may be derived with very small amounts of samples, and analysis may be completed within a short time of 3 to 12 hours.

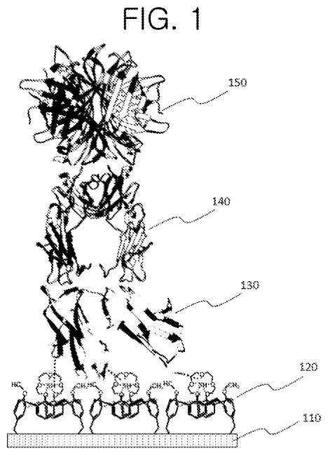

FIG. 1 schematically shows protein binding that is used in the screening method of the present invention. Referring to FIG. 1, in the method of screening a functional material according to the present invention, a substrate 110 is coated with a linker material 120 capable of immobilizing a capture protein 130, and the substrate 110 coated with the linker material 120 is treated with the capture protein 110, whereby the linker material 120 and the capture protein 130 bind specifically to each other. Then, the substrate is treated with a biotin-conjugated IgE 140, and the biotin-conjugated IgE 140 binds to the capture protein 130. Next, the substrate is treated with a fluorescently labelled streptavidin 150, and the fluorescently labelled streptavidin 150 binds to the biotin site. When all of the bindings are made to form the structure shown in FIG. 1, fluorescence is detected.

After the substrate is treated with the capture protein 130, the substrate is treated with a functional material candidate. When the functional material candidate inhibits the binding between the biotin-conjugated IgE 140 and the capture protein 130, the degree of fluorescence will decrease. Thus, scanning with a microarray scanner may be performed and the results may be analyzed. The fluorescence value resulting from the scanning process is expressed as a numerical value between 0 and 65535, which is a value of 2¹⁶.

The capture protein 130 may be FcεR1 that is able to bind specifically to IgE and affect histamine secretion on the surface of mast cells.

Hereinafter, the present invention will be described in more detail with reference to examples. These examples are intended to explain the present invention in more detail, and the scope of the present invention is not limited to these examples.

### Experimental Example 1

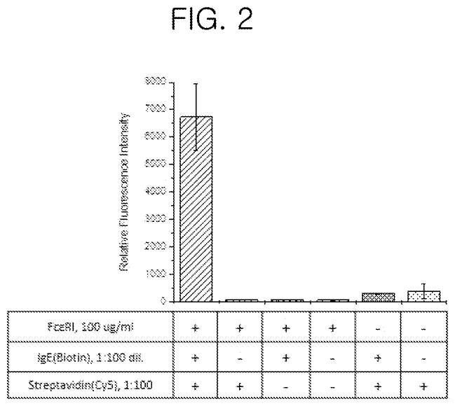

In order to confirm that the fluorescence characteristics are specifically detected in the structure of FIG. 1, substrates were treated with six combinations comprising one to three of 100 μg/ml FcεR1, biotin-conjugated IgE (1:100 diluted) and Cy5-labeled streptavidin (1:100 diluted), and the results are shown in FIG. 2 ((+): used, (−): not used).

Referring to FIG. 2, it was confirmed that, when the substrate was treated sequentially with 100 μg/ml FcεR1, biotin-conjugated IgE (1:100 diluted) and Cy5-labeled streptavidin (1:100 diluted) (the leftmost column in FIG. 2), high fluorescence was observed, but in the other cases, slight fluorescence caused by non-specific binding appeared, which was significantly very low.

Thus, it can be confirmed that fluorescence is detected when the structure shown in FIG. 1 is made.

Experimental Example 2

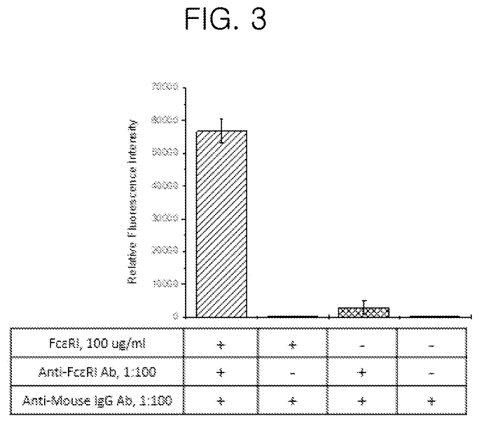

In order to confirm that FcεR1 was properly immobilized, substrates were treated with four combinations comprising one to three of 100 μg/ml FcεR1, anti-FcεR1 antibody (1:100 diluted) and anti-mouse IgG antibody (1:100 diluted), and the results are shown in FIG. 3 ((+): used, (−): not used).

Referring to FIG. 3, substrates were treated sequentially with FcεR1, anti-FcεR1 antibody specific to FcεR1, and anti-mouse IgG antibody (secondary antibody) specific to the anti-FcεR1 antibody, and whether fluorescence indicating binding to the anti-mouse IgG antibody was detected was checked. It was confirmed that, when the substrate was treated with all of the three substances (the leftmost column in FIG. 3), high fluorescence was observed, but in the other cases, slight fluorescence caused by non-specific binding appeared, which was significantly very low.

### Examples 1 to 28

FcεR1 diluted in 1×PBS (10% PEG-200, pH 7.4) buffer solution at a suitable concentration was added to a protein chip (in-house made) whose surface was coated with a linker material so that the inside of each well could be immobilized with protein. Then, the protein was immobilized with FcεR1 at 4° C. for 8 hours or more, washed twice with 1×PBST (0.5% Tween-20, pH 7.4) solution for 10 minutes, and blocked in 1% BSA (1×PBS, pH 7.4) solution for 1 hour while shaking at room temperature in order to block non-specific binding. Next, the protein chip was washed twice with 1×PBST (0.5% Tween-20, pH 7.4) solution for 10 minutes and rinsed with distilled water, and then water remaining in each well was removed using nitrogen gas. Among a list of natural materials, 0.5 μL of each of 28 extracts was spotted onto each well, and then 0.5 μL of biotin-conjugated IgE solution was spotted onto the same position, and the protein chip was placed in a wet box so as not to be dried and was incubated at room temperature for 1 hour. After the protein chip was washed and dried in the same manner as described above, 1 μg of Cy5-labelled streptavidin solution that strongly binds to the biotin was spotted onto each well, and the protein chip was placed in a wet box and incubated at room temperature for 1 hour.

### Comparative Example 1 (PC1)

The protein chip was treated in the same manner as in the Examples, except that anti-FcεR1 antibody and anti-mouse IgG antibody were used instead of the natural material extract, IgE and streptavidin.

### Comparative Example 2 (NC1)

The protein chip was treated in the same manner as in the Examples, except that 1.0 μL of DMSO buffer was used instead of 0.5 μL of the natural material extract and 0.5 μL of IgE.

### Comparative Example 3 (PC2)

The protein chip was treated in the same manner as in the Examples, except that 0.5 μL of DMSO buffer was used instead of 0.5 μL of the natural material extract.

### Experimental Example 3

Each of the protein chips of Comparative Example 1, Comparative Example 2, Comparative Example 3, and Examples 1 to 28 was scanned with a microarray scanner and the results were analyzed. From the results of the scanning, the fluorescence value appearing in each well was expressed as a numerical value between 0 and 65535, which is a value of 2¹⁶.

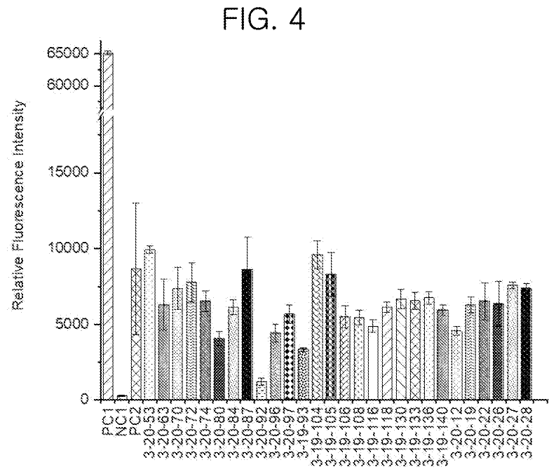

FIG. 4 is a graph showing the results according to Experimental Example 3. Referring to FIG. 4, Comparative Example 1 (PC1) is the intensity of fluorescence detected when using the anti-FcεR1 antibody specific to FcεR1 as primary antibody and the anti-FcεR1 antibody-specific anti-mouse IgG antibody as secondary antibody, Comparative Example 2 (NC1) is the level of fluorescence detected in the absence of IgE, and Comparative Example 3 (PC2) is the level of fluorescence detected when there was no artificial factor that interferes with the binding between IgE and FcεR1. Comparative Example 3 serves as a standard for the results of Examples 1 to 28 to be screened, and it is possible to evaluate the inhibitory effect against the binding between IgE and FcεR1 through relative comparison with Comparative Example 3.

In FIG. 4, it is confirmed that the natural materials of Example 9 (3-20-92), Example 12 (3-19-93), and Example 6 (3-20-80) have a higher effect of interfering with the binding between IgE and FcεR1 than other materials, and that Example 1 (3-20-53) and Example 13 (3-19-104) rather increase the binding between IgE and FcεR1, causing allergy, atopy and itching.

Although the present invention has been described above in detail with reference to the preferred embodiments, those skilled in the art will appreciate that the present invention can be variously modified and changed without departing from the spirit and scope of the present invention as set forth in the appended claims.

### DESCRIPTION OF REFERENCE NUMERALS

110: substrate, 120: linker material
130: capture protein, 140: biotin-conjugated IgE
150: Fluorescently labeled streptavidin

1. A method of screening a functional material, the method comprising steps of:
treating a linker material-coated substrate with a capture protein;
treating the capture protein-treated substrate with a functional material candidate;
treating the functional material candidate-treated substrate with biotin-conjugated IgE; and
treating the biotin-conjugated IgE-treated substrate with fluorescently labeled streptavidin.

2. The method according to claim 1, further comprising, after the step of treating the substrate with the capture protein, a step of treating the substrate with BSA to block non-specific binding.

3. The method according to claim 1, further comprising, before the step of treating the substrate with the fluorescently labeled streptavidin, a step of washing and drying the biotin-conjugated IgE-treated substrate.

4. The method according to claim 1, further comprising, before the step of treating the substrate with the functional material candidate, a step of washing and drying the capture protein-treated substrate.

5. The method according to claim 1, further comprising, after the step of treating the substrate with the fluorescently labeled streptavidin, a step of analyzing a degree of binding between the IgE and the capture protein.

6. The method according to claim 1, wherein the capture protein is FcεR1.
